(19) United States
(12) Patent Application Publication
(10) Pub. No.: US 2021/0231346 A1
(43) Pub. Date: Jul. 29, 2021

(54) HEATER

(71) Applicant: TekX Group, LLC, Parkersburg, WV (US)

(72) Inventors: John W. Florence, Parkersburg, WV (US); Joseph L. Florence, Parkersburg, WV (US)

(21) Appl. No.: 17/158,908

(22) Filed: Jan. 26, 2021

Related U.S. Application Data
(60) Provisional application No. 62/966,017, filed on Jan. 26, 2020.

(51) Int. Cl. F24H 3/04; A61L 9/03; A61L 9/18

(52) U.S. Cl. CPC F24H 3/0417; A61L 9/18; A61L 9/032

(57) ABSTRACT

A heater includes a housing, one or more infrared heating elements positioned inside the housing, and a fan positioned inside the housing. The housing has a front surface with a border around the perimeter of the front surface, with ventilation slots extending through the border, and an open central area. The housing has a rear surface with a border around the perimeter of the rear surface, with ventilation slots extending through the border, and a closed central area. The heater includes a removable first bezel to block the ventilation slots and the central area of the front and rear surfaces and a removable second bezel to block the ventilation slots on the border and being open in the central area. The heater may be used without the first or second bezels, with the first bezel alone, or with the second bezel alone.

### CROSS-REFERENCE TO RELATED APPLICATION

This application claims priority to U.S. Provisional Application No. 62/966,017, filed on Jan. 26, 2020, the disclosure of which is incorporated herein by reference in its entirety.

### FIELD

The technology described herein relates to a heater that is usable in three different modes, including a full convection mode, a combination of convection and radiation, and a full radiation mode.

### BACKGROUND

When you go outdoors on a sunny day, the heat generated by the sun “feels good.” This heat sensation has traveled millions of miles from the sun in the form of infrared energy. The benefit from the sun can also be produced by a heating unit that provides Far infrared energy.

Infrared energy is recognized as having three elements: Far Infrared, Medium Infrared, and Near Infrared, with each being categorized by their wavelength measurement. Far infrared (FIR) is a region in the infrared spectrum of electromagnetic radiation. Far infrared is often defined as any radiation with a wavelength of 15 micrometers (μm) to 1 mm (corresponding to a range of about 20 THz to 300 GHz), which places far infrared radiation within the CIE IR-B and IR-C bands.

FIR heat has many benefits. FIR heat penetrates the body to the inner layers of skin, muscle, and bones (by approximately 2-3 cm). FIR heat raises the temperature of body tissues, which provides a deep, prolonged heat. FIR heat increases oxygenation and regeneration of blood. Other health benefits associated with FIR heat include: raising temperature in body tissue; expanding capillary vessels; rejuvenating cells; promoting blood circulation without raising blood pressure; helping to remove toxins from cells; faster recovery of muscle strains; reduction in the effect of allergic rhinitis (commonly known as hay fever); improves elasticity of skin and helps to reduce wrinkles; reduces pain in hands or feet that is associated with arthritis or tendonitis; and reduces pain associated with diabetic neuropathy and poor circulation, among other benefits.

### SUMMARY

A tri-mode heater that provides both radiation and convection modes is disclosed and described.

### BRIEF DESCRIPTION OF THE DRAWING FIGURES

FIG. 1 depicts a perspective front view of a heater according to the invention, shown without bezels 1 and 18 in place;

FIG. 2 depicts a perspective front view of the heater of FIG. 1 in the combined convection and radiation modes, and having bezel 1 attached to the backside of the heater 16;

FIG. 3 depicts a perspective rear view of the heater of FIG. 1 with bezel 1 illustrated, but not yet attached to the heater 16. This figure also illustrates the exhaust vent insert 3 as having an optional wood grain finish;

FIG. 4 depicts an exploded view of the heater of FIG. 1. This view, consistent with FIG. 1, does not include bezels 1 and 18;

FIG. 5 depicts an exploded view of the heater of FIG. 3;

FIG. 6 depicts a perspective view of the heater of FIG. 1 having attached a wood-grained finished bezel 18 installed on a short adjustable stand;

FIG. 7 depicts a perspective view of the heater of FIG. 1 having attached a wood-grained finished bezel 18 installed on a tall adjustable stand;

FIG. 8 depicts a perspective view of additional features of the claimed invention showing modularity features of the design; and

FIG. 9 depicts a perspective view of the design of FIG. 8 showing the modular parts installed together.

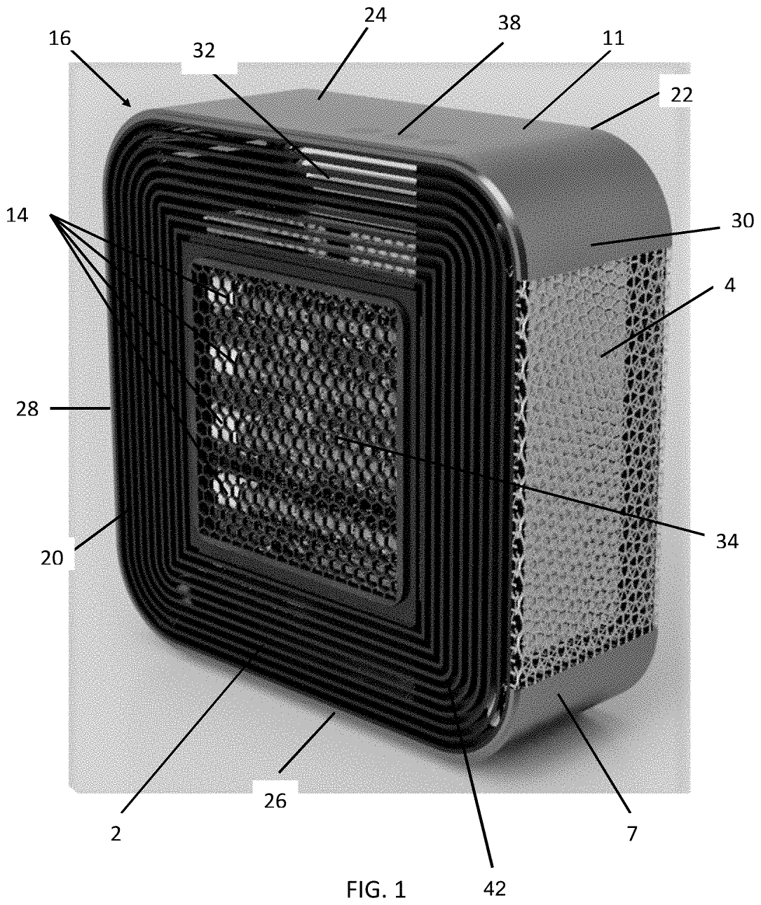

### DETAILED DESCRIPTION

The technology described herein relates generally to a heater 16, such as a personal heater or a room heater. The heater 16 is a dual-stage, localized space heater with three modes of operation. These modes include:

1) convection only;

2) combination of convection and radiation; and

3) radiation only.

The heater 16 is unique in that it can provide the normal benefit of heating a room. In addition, it provides the additional benefit of FIR heat therapy directed at specific parts of a user's body, such as the feet, face, hands, or other body parts. The heater has three operating modes that provide: convection heat only, FIR only, or a combination of both convection heat and FIR. The different modes of operation are provided by the selection and placement location of two bezels that are applied to surface of the heater 16. There is presently no known heaters on the market that offer this combination of features.

The heater 16 may be modular, if desired. For example, the heater may include a heater module 50, an air purification module 60, a power module 70, and a fan module 80, combinations of these modules, or other modules, as desired. The provision of modularity provides the consumer with the option of selecting different modules based upon individual needs, as will be discussed in greater detail below. The various modules may be joined together in any known manner, such as by being screwed together or other connecting means.

The heater 16 also incorporates a stand. The stand makes it possible to aim the heater 16 at different body parts when the FIR mode is utilized. The stand may be adjustable so that a user can position it at different heights. In addition, the stand permits the user to change the angle of the heater 16 to further aim it at body parts.

The device includes a heater unit 16, a first removable bezel 1, and a second removable bezel 18. The three modes are possible due to the use of the two removable bezels 1, 18 that can be attached to cover either the front or rear sides of the heater 16. The bezels 1, 18 are only used one at a time. Both bezels 1, 18 are not used at the same time.

The heater 16 shown is substantially rectangular in shape. It includes a front side 20, a rear side 22, a top side 24, a bottom side 26, a left side surface 28 and a right-side surface 30. Both the front 20 and rear 22 sides of the heater 16 have a border area 42 that surrounds the entire front and rear surface 20, 22 of the heater 16. The border 30 is made of a hard plastic or metal material. Ventilation slots 32 extend through both the front side surface 20 and the rear side surface 22. These slots 32 permit convection heated air to exit through the ventilation slots 32. A central area 34 on the front surface 20 of the heater 16 exposes internal heating elements 14 that radiate heat through the central area 34 of the heater 16. A central area 36 on the rear side 22 of the heater 16 is closed by an exhaust vent insert 3.

The heater 16 includes infrared heating elements 14 that create an infrared heat source. The infrared heating elements 14 are positioned in a central part of the interior of the heater 16, between the four sides 24, 26, 28, 30. The infrared heating elements 14 emit FIR energy. The infrared elements 14 may be four infrared radiant bulbs. The bulbs 14 are positioned adjacent the central window 34 on the front side 20 of the heater 16. The heater 16 also includes a heat exchanger 12, which is generally made of a good metal conductor, such as a copper heat chamber. The heater 16 also includes one or more fans 5 that are used to move air onto and across the heat exchanger 12 to create heated air flow. In the embodiment disclosed herein, three axial fans 5 having 90 mm diameters are utilized. Other sized fans and any number of fans may be utilized. The fans 5 circulate air inside the heater 16 and force the air out of the heater 16, either through the front or rear sides 20, 22 of the heater 16.

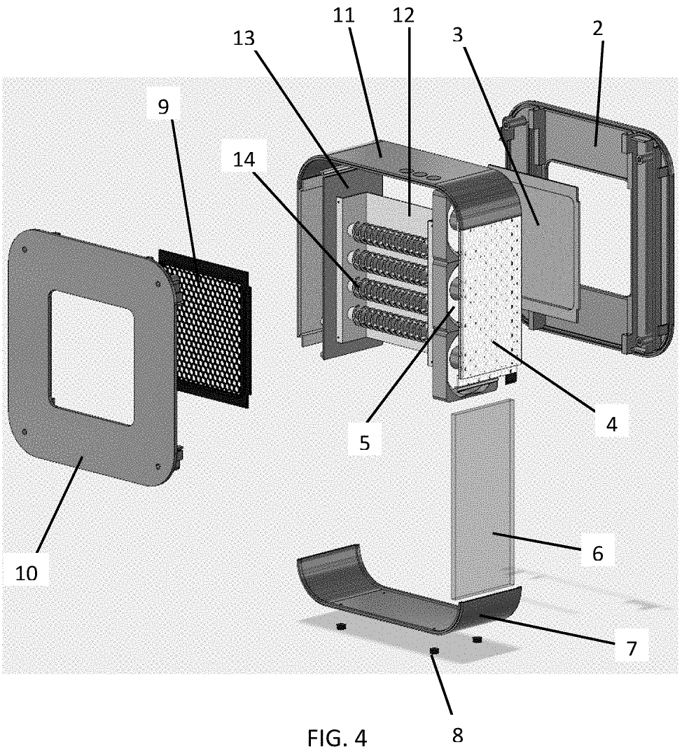

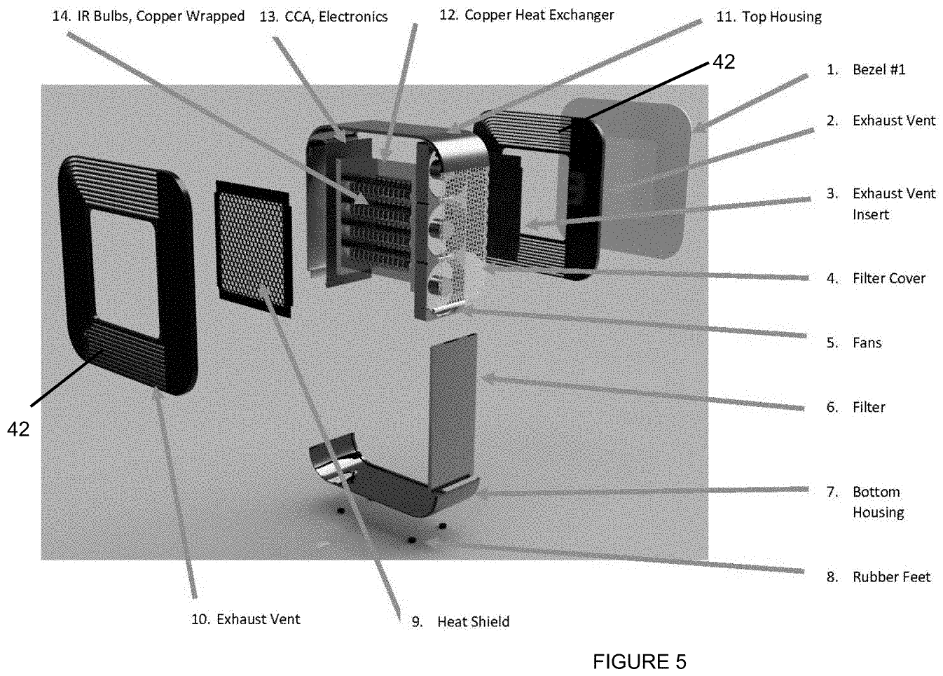

The internal elements of the heater 16 are shown in FIGS. 4 and 5, with the difference between the two figures being that FIG. 5 shows the first bezel 1 positioned adjacent the rear surface 22 of the heater 16. The exhaust vent 2 forms the rear surface 22 of the heater 16. The exhaust vent 2 includes the exhaust vent insert 3 that blocks the central area 36 of the rear surface 22. A filter 6 is positioned on the right side 30 of the heater 16. The filter 6 is positioned adjacent three (3) fans 5 and the filter 6 is covered by a filter cover 4, which forms the right-side wall 30 of the heater 16.

The unit 16 includes a bottom housing 7 that has feet 8. The front side 20 of the heater 16 includes an exhaust vent 10 and a heat shield 9. The rear and front exhaust vents 2, 10 include vent slots 32 that permit air to be exhausted from the heater 16. The heater 16 includes a top housing 11 that forms the top side 24 and left side 28 of the heater housing. The interior of the heater includes IR bulbs that may or may not be copper wrapped 14, CCA electronics 13, and a copper heat exchanger 12. Other types of wrappings may be used on the bulbs, if desired. The heat exchanger 12 surrounds three sides of the IR bulbs and the CCA electronic panel is positioned between the heat exchanger 12 and the left side 28 of the top housing 11.

Activation members 38, such as buttons are shown positioned on the top side 24 of the heater 10. These buttons 38 are used to turn the heater 16 on and to select the temperature level of the heater.

The first removable bezel 1, with the glass insert, when attached to the back side 22 of the heater 16, is used to provide the combined convection and radiation modes exiting from the front side 20 of the heater. Alternatively, bezel 1, when attached to the front side 20 of the heater 16, is then positioned to provide the convection only mode exiting from the back side 22 of the heater 16. The second removable bezel 18, with the open center, when placed on the front side 20 of heater 16, is used to provide the radiation only mode exiting from the front side 20 of the heater 16. The dual mode is used to provide the convection only mode and the second removable bezel 18 is used to provide the radiation only mode. The dual mode or first bezel 1 resembles a picture frame that has an outer border and a glass insert in the middle of the border. The border substantially matches the border on the front or rear side of the heater 16. The border material of the bezel is a hard plastic or glass material that may be opaque or transparent. The glass insert is transparent and does not permit FIR energy to travel through the glass insert. The glass insert blocks or shields the FIR energy.

The second removable bezel 18 is used to provide the radiation only mode. The bezel resembles a picture frame, like that of the first bezel 1, but the center part of the bezel is open and not covered by glass. The outer part of the bezel is made of a solid material and substantially matches the shape of the border on the front side of the heater.

The bezels 1, 18 may be attached or mounted to the heater in any known manner. The bezels 1, 18 may be attached, for example, by magnets, snap-on hardware, screws, or clips.

To use the device 16 in the convection only mode, the first bezel 1 is attached to the front side 20 of the heater 16. When attached to the front side 20 of the heater 16, the first bezel 1 covers the ventilation slots 32 on the front side 20 and the central part of the front side 20 where radiant heat is emitted. This forces all convection heated air to exit the unit from the ventilation slots 32 on the back side 22 of the device 16. When positioned on the front side 20 of the heater 16, the central glass element of the first bezel 1 blocks the infrared energy from leaving the heater 16. This blocks radiant heat and, instead, the radiant energy is transferred to the heat exchanger, which then heats the air in unit 16 as it moves across the heat exchanger surface. The air exits through the ventilation slots 32 in the border area of the back side 22 of the heater 16.

To use the device 16 in both convection and radiant heat mode, the first bezel 1 needs to be positioned on the rear side 22 of the heater 16. By doing so, the ventilation slots 32 on the back surface 22 are blocked. When the heater 16 is operational, convection heat flows from the ventilation slots 32 on the front side 20 of the heater 16 while radiant heat emits from the now fully exposed FIR elements 14.

To use the device 16 in radiant heat mode only, the user removes the first bezel 1 from the heater 16 and installs the second bezel 18 on the front side 20 of the heater 16. Because the second bezel 18 does not have glass in the center of the bezel, the FIR elements 14 are exposed. This permits the FIR to be directed towards the user while blocking the front ventilations slots 32 and forcing convection air out through the rear ventilation slots 32 on the back side 22 of the heater 16. Some convection air will still flow out of the front opening, but most of the air will travel through the rear ventilation slots 32. The air that flow through the front opening helps to cool the infrared bulbs 14.

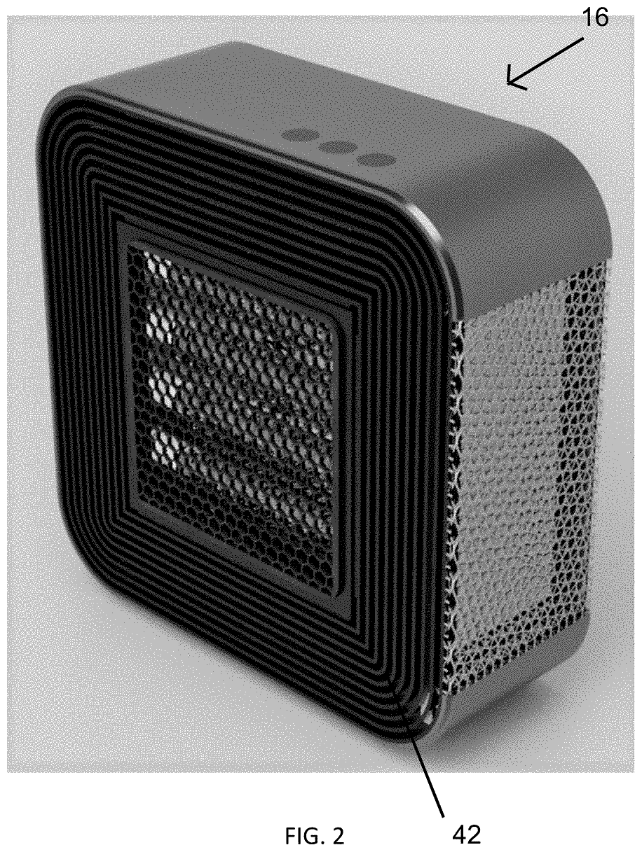

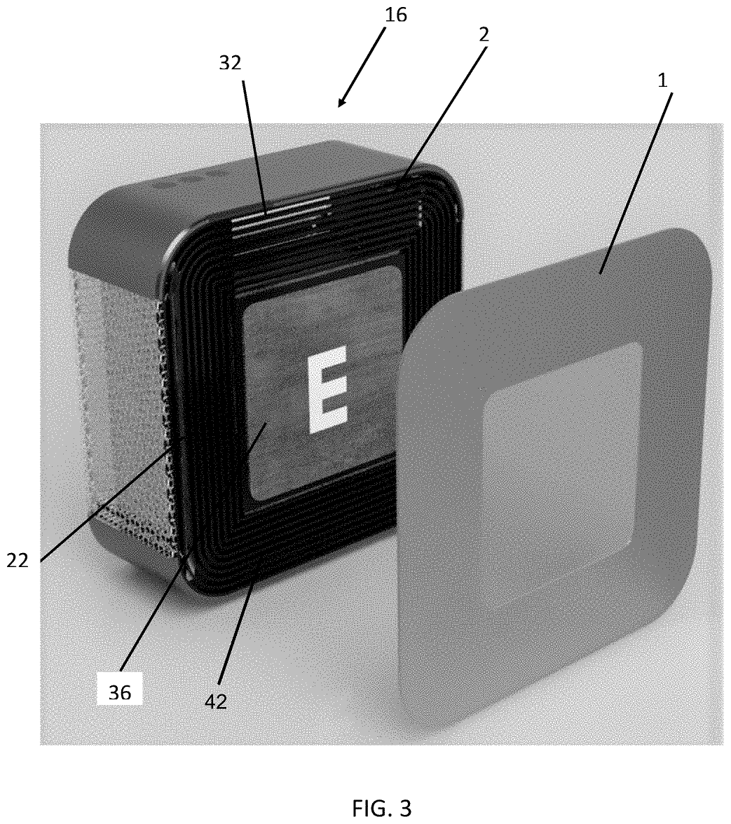

Referring to FIGS. 2 and 3, the convection and radiation modes are shown. In this embodiment, the heater 16 shows a plurality of ventilation slots 32 extending through the border area of the front side 20 of the heater 16. The central part of the front side 20 of the heater 16 shows four infrared bulbs 14 positioned behind a heat shield 9 in the form of a protective grid. This heat shield 9 permits heat to emanate from the infrared bulbs 14 but does not permit a user to touch the bulbs 14 and potentially be burned. The top side 24 of the heater 16 shows three activation buttons 38. The right side 30 of the heater 16 shows a filter cover 4 that is in the form of a metal mesh material that permits air to enter the heater 16 through the side of the heater 16. Fans 5, while not shown in this figure, are positioned behind the filter 6 and filter cover 4. In this embodiment, the first bezel 1 is applied to the rear side of the heater 16, forcing all convective air to exit through the front of the heater 16. At the same time, radiant heat is emitted from the infrared bulbs 14.

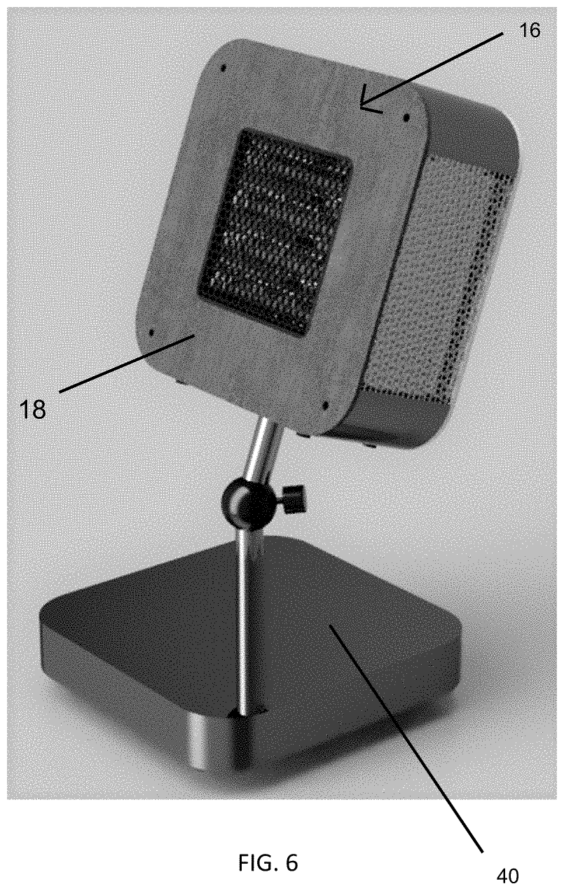

FIGS. 6 & 7 depict the radiant mode of the heater 16. In this mode, the second bezel 18 is positioned over the front surface of the heater 16. The second bezel 18 covers the ventilation slots 32 in the border of the front surface. The central part of the heater 16 remains open to permit radiant heat to emanate from the infrared bulbs 14.

In convection only mode, which is not shown, the user places the first bevel 1 over the front side of the heater 16. This permits all heat and air to exit through the rear side of the heater 16 through the ventilation slots 32 in the border area of the rear side.

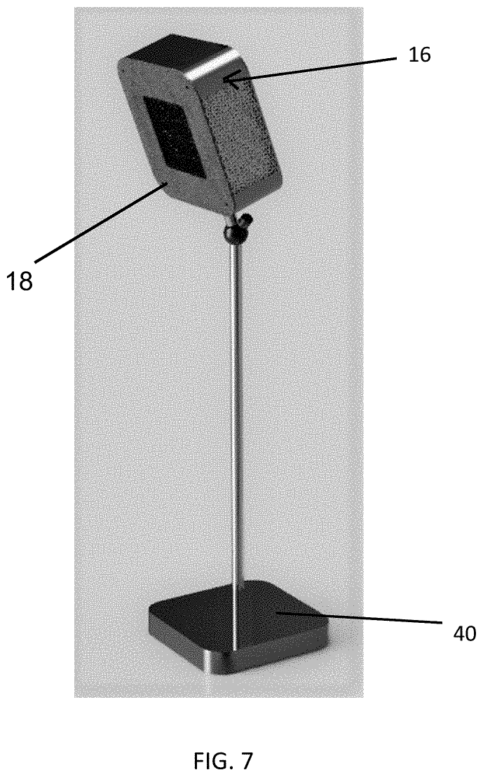

An adjustable stand 40 and/or tripod may be provided with the heater 16. FIGS. 6 and 7 depict the heater 16 installed on two different types of adjustable stands 40. The adjustable stands 40 are advantageous because they allow a user to raise or lower the heater 16 into a desired position to direct the Radiant or Convective heat onto a specific location selected by the user. The heater/stand 16, 40 interface also includes a tilt feature that allows the user to lean the heater downward or upward, providing even greater control in directing the chosen heat energy to a specific location. The stand 40 or tripod allows the user to position the heater 16 directly where they want to focus the heat. The user has the option to focus direct exposure of the FIR energy emitted from the heater 16 onto specific portions of their body. FIG. 6 depicts a heater 16 mounted to a stand, in a downward tilted position. The stand is height adjustable upwardly and downwardly from 10″ to 50″. In another embodiment, the stand is height adjustable upwardly and downwardly from 20″ to 36″. FIG. 7 depicts a heater 16 mounted to a stand 40, in a lowered position, with the heater 16 tilted backward. The heater 16 may be directly attached to the stand 40 or tripod or may be operated independent from the stand 40 or tripod.

The first bezel 1 may be made of powder coated carbon steel with a glass insert. The powder coated carbon steel forms the border of the first bezel 1. The second bezel 18 may be made of powder coated carbon steel.

The exhaust vent 2 may be made of a plastic material, such as a hard-plastic material. The exhaust vent insert 3 may be made of plastic, metal, wood, or other materials. The filter cover 4 may be made of powder coated carbon steel. The fans 5 may be made of plastic or metal. The filter 6 may be made of foam or a HEPA like material. The bottom housing 7 may be made of powder coated carbon steel. The rubber feet 8 may be made of rubber or plastic materials. The heat shield 9 may be made of carbon steel. The heat shield 9 may be coated with a heat protective flocked coating. This coating may help to prevent burns. The exhaust vent 10 may be made of a plastic material. The top housing 11 may be made of powder coated carbon steel, of brushed or anodized aluminum, or of plastic. The copper heat exchanger 12 may be made of copper. The CCA (Circuit card assembly) electronics 13 comprise an assembled circuit board, as known by those of skill in the art. Other materials may alternatively be used, as known by those of skill in the art.

The first and second bezels 1, 18 fit over the front or rear surfaces 20, 22 of the heater 16 and can have different shapes depending upon the configuration and shape of the heater 16. The heater 16 may be a 1500-watt space heater. The heater 16 can stand alone on any flat surface, such as a floor or tabletop, or can be placed on a tripod or stand 40. Physical dimensions of the device 16 may be about 13.25 inches in height, 13.25 inches in width, and 4 inches in depth. Physical dimensions may be smaller or larger, such as about 2 inches to about 60 inches in both width and height, and 1 inch to 30 inches in depth. The heater 16 may be other shapes, other than those shown.

The invention enables the user to customize the heat source. A convection heater 16 eventually heats an entire room, providing a warm environment for everyone in the room. Radiant heat will only warm the person to whom the IR energy is directed. This will allow a room to remain at a lower temperature, while heating a single person.

While not shown, the bezel 1, 18 selection may be performed automatically. For example, bezels 1, 18 may be stored in the heater 16 and may be pneumatically or electronically applied to the surfaces of the heater 16. The heater 16 may use alternative heat sources, such as ceramic, as well as alternate heat energy shields and alternate air shielding.

As previously discussed, the heater may include various modules that are separable from one another and removable from one another. The modules may be optional or may be required for the heater to work. The heater module 50 may be used with the fan module 80 and the power module 70 only, if desired, to create a base model of the heater. Additional modules can be added, such as an air purification module 60, an IR module, or other modules.

The fan module 80 is provided to move air through the heater. It provides air circulation to support all combinations of additional modules. The fan may be a single large fan, several independent smaller fans, or complex dual stage fans. Any type of fan configuration may be used, as desired, such as axial, crossflow, circumferential, or other types of fans.

The power module 70 is the central module that connects to an external power source, such as a home wall outlet. The power module 70 is designed to provide enough power to operate the heater module 50 and all other modules that are connected to the heater module 50. The power module 70 may also include connectivity features, a user interface, and electronics, such as a control system, wiring, and the like. Connectivity features may include WIFI, Bluetooth, or the like. The power module 70 may include electronics that permit the user to access the heater's controls remotely, such as by using a remote. The electronics may permit the user to access the heater using their mobile device, such as with a mobile phone, mobile station, or other device.

The air purification module 60 may employ active and/or passive air purification methods. These methods may include HEPA filtration, ultraviolet lights, ozone generators, carbon filters, photocatalyst (PCO's) or any other type of air purification device or process. The air purification module 60 may be a single module that offers possible pairings or combinations of air purification methods. Alternatively, the air purification module 60 may include separate independent modules that may be coupled together to provide modules that are selectively created for each individual need and to provide unique air purifications modules 60.

The heater module 50, or IR module (infrared module), is the module that provides the heating function. The heating module includes the Far Infrared Radiation (FIR) bulbs. The heater module 50 may also include ceramic heating elements, Positive Temperature Coefficient (PTC) heating elements, or other such elements. Like the air purification module 60, the heater module 50 may be provided in multiple configurations. For example, the FIR heater module 50 may be used for healing benefits and a PTC module may be used for zone heating. Each module may be considered a heater module 50 and these modules may be coupled together to form a unitary heater module 50 that is made up of multiple modules. The modules may be joined in any desired way, such as by screwing the modules together to form a single module. Alternatively, the modules may be pressed together using protrusions and receptacles, where protrusions are positioned on one surface for joining with receptacles on an adjacent surface. Other ways to join the modules together are also anticipated.

Other modules may include a cooling module or an aromatherapy/essential oils module. A cooling module is utilized to provide passive and/or active cooling. This module may be attached to the fan module 80, for example. When the cooling module is used in conjunction with the fan module 80, the device may provide a zone of air conditioning and serve as a cooling unit for cooling a user or for cooling an area.

An aromatherapy/essential oils module may provide passive and/or active methods that provide an environmental aroma output, which can be selected by a user for personal enjoyment and/or for therapeutic health treatment. The aromatherapy module may include a method of dealing with chronic illness pain relief.

Each module, and combinations thereof, when attached to the heater, power, and fan modules 50, 70, 80 are self-standing, such as stably seating on a surface. Alternatively, the modules and/or combinations thereof can attach to the stand 40, as discussed above, to ensure optimum placement by a consumer. Each of the modules 50, 60, 70, 80 shall be PLUG-AND-PLAY (PnP). PnP devices are those that work with a computer system as soon as they are connected.

Alternative modules, not mentioned above, may alternatively be used that are presently envisioned or not yet envisioned. For example, the Alternative modules may include a light therapy module, a music therapy module, a voice therapy module, or other modules that offer comfort features or therapeutic benefits to the consumer.

The various modules may be used in any desired combination to provide multiple benefits to the consumer. The design provides the consumer with flexibility purchase only the base modules (e.g., the power module 70 and fan module 80), and then add other modules/features to provide comfort and/or therapeutic benefits.

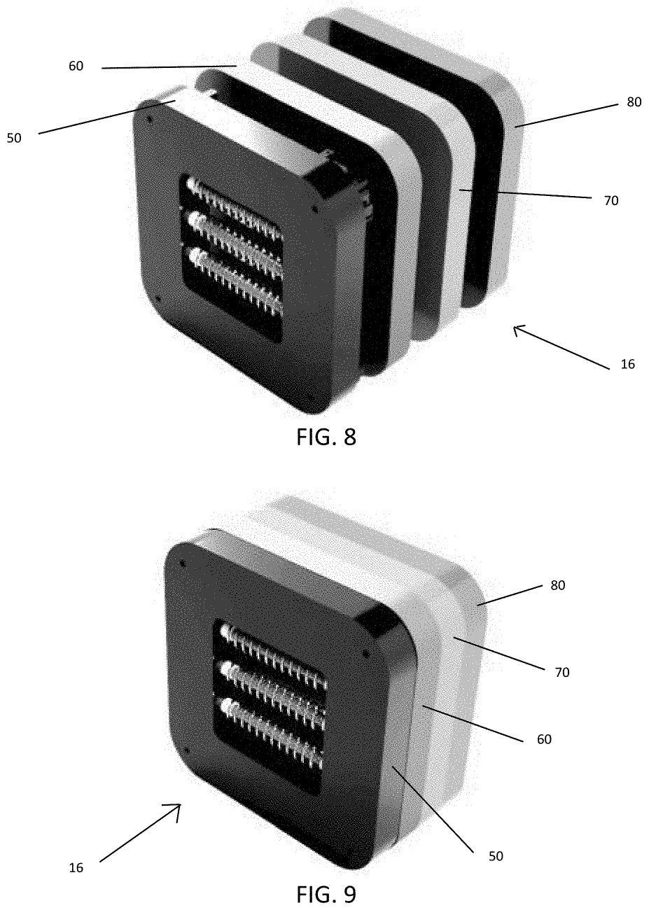

FIGS. 8 and 9 depict the modular aspects described above. The modules depicted include a heater module 50, a power module 70, an air purification module 60, and a fan module 80. Each of the modules are shown as being of similar exterior shape such that when the modules are joined together, they form a common outer boundary. In particular, the base of the device is shown as being substantially flat to permit the device to seat on a flat surface. Alternatively, the various modules may have different shapes and sizes, with some of the modules being smaller or larger than other modules. It is preferred that the base of the module permits the device to seat on a surface. However, this does not require that all modules be the same size and shape.

FIG. 8 depicts the various modules being separated from one another but aligned with each other. FIG. 9 depicts the various modules being coupled together. Other modules may be provided, as desired, based upon consumer interests and needs.

According to a first embodiment, a heater includes a housing, one or more infrared heating elements, and a fan. The one or more infrared heating elements are positioned inside the housing. The fan is positioned inside the housing. A front surface of the housing has a border around the perimeter of the front surface, with ventilation slots extending through the border, and an open central area. A rear surface of the housing has a border around the perimeter of the rear surface, with ventilation slots extending through the border, and a closed central area. A removable first bezel is sized to seat on the front and rear surface of the housing and to block the ventilation slots and the central area of the front and rear surfaces. A removable second bezel is sized to seat on the front surface of the housing and to block the ventilation slots on the border and being open in the central area. The heater may be used without the first or second bezels, with the first bezel alone, or with the second bezel alone.

The heater may also include a heat exchanger positioned at least in part around the one or more infrared heating elements. The fan may be positioned on one side of the housing. The heater may also include a filter positioned adjacent the fan and a filter cover positioned over the filter, with the filter cover forming a side wall of the housing. The filter cover can be a metallic mesh. The first bezel may be made of plastic and glass and the second bezel may be made of plastic. The heater may also include an activation means for activating the heater.

In another embodiment, a modular FIR heater system includes a heater module, a power module, and a fan module. The heater module has FIR heating components. The power module has at least electronics. The fan module has a least one fan. The heater, power and fan modules are coupled together to form a heater.

The heater system may also include an air purification module. The heater system may also include one or more of a cooling module, an aroma therapy module, an essential oils module, a light therapy module, a music therapy module, and a voice module. The heater system may include plug-and-play technology. The power module may include wireless connectivity. The wireless connectivity may be Bluetooth. The power module may include a user interface. The heater module may include Far Infrared Radiation (FIR) bulbs. The heater module may also include one or more of ceramic heating elements and positive temperature coefficient heating elements.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What is claimed is:

1. A heater comprising:
a housing,
one or more infrared heating elements positioned inside the housing;
a fan positioned inside the housing;
a front surface of the housing having a border around the perimeter of the front surface, with ventilation slots extending through the border, and an open central area;
a rear surface of the housing having a border around the perimeter of the rear surface, with ventilation slots extending through the border, and a closed central area;
a removable first bezel sized to seat on the front and rear surface of the housing and to block the ventilation slots and the central area of the front and rear surfaces; and
a removable second bezel sized to seat on the front surface of the housing and to block the ventilation slots on the border and being open in the central area,
wherein the heater may be used without the first or second bezels, with the first bezel alone, or with the second bezel alone.

2. The heater of claim 1, further comprising a heat exchanger positioned at least in part around the one or more infrared heating elements.

3. The heater of claim 1, wherein the fan is positioned on one side of the housing, and further comprising a filter positioned adjacent the fan and a filter cover positioned over the filter, with the filter cover forming a side wall of the housing.

4. The heater of claim 4, wherein the filter cover is a metallic mesh.

5. The heater of claim 1, wherein the first bezel is made of plastic and glass and the second bezel is made of plastic.

6. The heater of claim 1, further comprising activation means for activating the heater.

7. A modular FIR heater system comprising:
a heater module having FIR heating components;
a power module having at least electronics; and
a fan module having a least one fan,
wherein the heater, power and fan modules are coupled together to form a heater.

8. The heater system of claim 7, further comprising an air purification module.

9. The heater system of claim 7, further comprising one or more of a cooling module, an aroma therapy module, an essential oils module, a light therapy module, a music therapy module, and a voice module.

10. The heater system of claim 7, wherein the heater system comprises plug-and-play technology.

11. The heater system of claim 7, wherein the power module includes wireless connectivity.

12. The heater system of claim 11, wherein the wireless connectivity is Bluetooth.

13. The heater system of claim 7, wherein the power module includes a user interface.

14. The heater system of claim 7, wherein the heater module includes Far Infrared Radiation (FIR) bulbs.

15. The heater system of claim 14, wherein the heater module further includes one or more of ceramic heating elements and positive temperature coefficient heating elements.
